SEQUENCE LISTING (10 sequences)

SEQ ID NO: 1 - protein, 135 aa, Artificial Sequence
  RLYB212 Heavy Chain Variable Region
QVQLQQSGPGLVKPSQTLSLTCAISGDSVSSNSAAWNWIRQSPSRGLEWLGRTYFRSNWYNDYAASVKSRITINQDTSKNQLSLQLNSVTPEDTAVYYCARDGAWGGSSWWPGLPHHYYSGMDVWGQGTTVTVSS

SEQ ID NO: 2 - protein, 108 aa, Artificial Sequence
  RLYB212 Light Chain Variable Region
EIVLTQSPATLSLSPGERATLSCRASQSVSSYLAWYQQKPGQAPRLLIYDASKRATGIPARFSGSGSGTDFSLTIRSLEPEDFAVYYCQQRSDWQGLTFGGGTKVEIK

SEQ ID NO: 3 - protein, 10 aa, Artificial Sequence
  RLYB212 Variable Heavy Complementarity Determining Region 1
GDSVSSNSAA

SEQ ID NO: 4 - protein, 9 aa, Artificial Sequence
  RLYB212 Variable Heavy Complementarity Determining Region 2
TYFRSNWYN

SEQ ID NO: 5 - protein, 25 aa, Artificial Sequence
  RLYB212 Variable Heavy Complementarity Determining Region 3
ARDGAWGGSSWWPGLPHHYYSGMDV

SEQ ID NO: 6 - protein, 6 aa, Artificial Sequence
  RLYB212 Variable Light Complementarity Determining Region 1
QSVSSY

SEQ ID NO: 7 - skipped

SEQ ID NO: 8 - protein, 10 aa, Artificial Sequence
  RLYB212 Variable Light Complementarity Determining Region 3
QQRSDWQGLT

SEQ ID NO: 9 - protein, 465 aa, Artificial Sequence
  RLYB212 Heavy Chain
QVQLQQSGPGLVKPSQTLSLTCAISGDSVSSNSAAWNWIRQSPSRGLEWLGRTYFRSNWYNDYAASVKSRITINQDTSKNQLSLQLNSVTPEDTAVYYCARDGAWGGSSWWPGLPHHYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  RLYB212 Light Chain
EIVLTQSPATLSLSPGERATLSCRASQSVSSYLAWYQQKPGQAPRLLIYDASKRATGIPARFSGSGSGTDFSLTIRSLEPEDFAVYYCQQRSDWQGLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0141066 A1
(43) Pub. Date: May 2, 2024

(54) Method of Administering Anti-HPA-1a Monoclonal Antibody

(71) Applicant: Rallybio IPA, LLC, New Haven, CT (US)

(72) Inventor: Douglas L. Sheridan, Branford, CT (US)

(21) Appl. No.: 18/575,804

(22) PCT Filed: Jun. 30, 2022
(86) PCT No.: PCT/US2022/035677

§ 371 Date: Dec. 29, 2023

Related U.S. Application Data
(60) Provisional application No. 63/217,636, filed on Jul. 1, 2021.

(51) Int. Cl. C07K 16/34; A61P 37/06

(52) U.S. Cl. CPC C07K 16/34; A61P 37/06

(57) ABSTRACT

Methods are described for administering to a subject an anti-human platelet antigen (HPA)-1a monoclonal antibody. The methods include parenterally administering to the subject a pharmaceutical composition comprising an anti-HPA-1a monoclonal antibody in an amount of about 0.02 ng/mL to about 0.4 ng/mL, and/or an amount effective to achieve a maximum plasma concentration of the anti-HPA-1a monoclonal antibody of about 0.01 IU/mL to about 10 IU/mL in the subject.

### CROSS-REFERENCE TO RELATED APPLICATIONS

This application claims the benefit of priority of U.S. Provisional Patent Application No. 63/217,636, filed on Jul. 1, 2021.

### BACKGROUND

Fetal and neonatal alloimmune thrombocytopenia (FNAIT) is a bleeding disorder that is caused by an attack by the mother's immune system on her fetus' platelets. This response by the mother's immune system stems from the fetus' platelets having proteins on their surface that are inherited from the father but absent from the mother's platelets. If fetal platelets enter the mother's circulation, the mother's immune system produces antibodies to fight against the fetal platelets in an alloimmune response. These antibodies can pass through the placenta and bind to and destroy the fetus' platelets, resulting in fetal thrombocytopenia.

Fetal-maternal incompatibility in the human platelet antigen (HPA)-1 locus is the most common cause of FNAIT, accounting for 85% to 90% of severe FNAIT cases (Eksteen et al. 2015). Such incompatibility is due to a single nucleotide polymorphism that results in a leucine/proline polymorphism at residue 33 of integrin β3 (glycoprotein IIIa), which is present in the platelet membrane and on endothelial cells (Newman et al. 1989). FNAIT results where the mother is negative for the HPA-1a antigen (HPA-1b homozygous), and the fetus has HPA-1a positive cells inherited paternally (Bussel 2009).

The propensity to develop HPA-1a antibodies is closely linked to a certain human leukocyte antigen (HLA) type (Kjeldesen-Kragh et al. 2007). The risk for women who are HPA-1a negative to become HPA-1a immunized is approximately 25 times higher in those who are positive for the HLA-DRB3*01:01 allele than in women who do not carry this allele (Kjeldesen-Kragh and Olsen 2019).

The consequences of FNAIT can be severe. While in some cases there are no symptoms, fetuses and newborns with FNAIT can experience mucosal bleeding, hematomas, retinal bleeding, and intracranial hemorrhage (ICH), which may lead to intrauterine death or lifelong disability (Radder et al. 2003; Kjeldesen-Kragh and Skogen 2013).

Yet, there is no accepted treatment available for the prevention of alloimmunization in HPA-1a negative women. For women with known pregnancies at risk for developing FNAIT (women who previously gave birth to an FNAIT-affected child), the general approach is weekly maternal intravenous administration of human immune globulin (IGIV), with or without the addition of glucocorticoids, but there is no consensus as to the optimal dosing strategy. Moreover, IGIV does not prevent alloimmunization and, in the doses administered, is accompanied by reports of poor tolerability (Vitiello et al. 2019; Rossi et al. 2015).

Thus, there remains a need for an effective treatment to prevent alloimmunization to HPA-1a-positive platelets in the mother.

### SEQUENCE LISTING

This application contains a Sequence Listing, which has been submitted electronically in ASCII format. The ASCII copy was created on Jun. 20, 2022, is named IPA_007_WO1_SL.txt, and is 9,969 bytes in size.

### SUMMARY OF THE INVENTION

Some of the main aspects of the present invention are summarized below. Additional aspects are described in the Detailed Description of the Invention, Example, and Claims sections of this disclosure. The description in each section of this disclosure is intended to be read in conjunction with the other sections. Furthermore, the various embodiments described in each section of this disclosure can be combined in various ways, and all such combinations are intended to fall within the scope of the present invention.

Accordingly, the disclosure provides a method of administering an anti-HPA-1a monoclonal antibody to a subject that is HPA-1a negative, in which the method comprises administering to the subject a pharmaceutical composition comprising the anti-HPA-1a monoclonal antibody. The disclosure also provides a pharmaceutical composition comprising an anti-HPA-1a monoclonal antibody for use in a method of administering the anti-HPA-1a monoclonal antibody to a subject that is HPA-1a negative. The anti-HPA-1a monoclonal antibody comprises (i) a variable heavy (VH) complementarity determining region (CDR) 1 having the amino acid sequence of SEQ ID NO: 3; (ii) a VH CDR2 having the amino acid sequence of SEQ ID NO: 4; (iii) a VH CDR3 having the amino acid sequence of SEQ ID NO: 5; (iv) a variable light (VL) CDR1 having the amino acid sequence of SEQ ID NO: 6; (v) a VL CDR2 having the amino acid sequence of SEQ ID NO: 7; and (vi) a VL CDR3 having the amino acid sequence of SEQ ID NO: 8. In some embodiments, the anti-HPA-1a monoclonal antibody comprises a heavy chain variable region having the amino acid sequence of SEQ ID NO: 1, and a light chain variable region having the amino acid sequence of SEQ ID NO: 2.

The pharmaceutical composition is administered parenterally. In certain embodiments, the pharmaceutical composition is administered subcutaneously.

In some embodiments, the anti-HPA-1a monoclonal antibody is administered in an amount of about 0.015 mg to about 0.6 mg, or about 0.05 mg to about 0.35 mg. In certain embodiments, the anti-HPA-1a monoclonal antibody is administered in an amount of about 0.1 mg to about 0.29 mg.

In some embodiments, the anti-HPA-1a monoclonal antibody is administered in an amount effective to achieve a maximum plasma concentration of the anti-HPA-1a monoclonal antibody of about 3 ng/mL to about 40 ng/mL, or about 5 ng/mL to about 30 ng/mL, in the subject. In certain embodiments, the anti-HPA-1a monoclonal antibody is administered in an amount effective to achieve a maximum plasma concentration of the anti-HPA-1a monoclonal antibody of about 6 ng/mL to about 10 ng/mL in the subject.

In certain embodiments, the anti-HPA-1a monoclonal antibody is administered in an amount effective to achieve a maximum plasma concentration of the anti-HPA-1a monoclonal antibody of about 0.45 IU/mL.

In some embodiments, the subject is an HPA-1a negative woman. In certain embodiments, the subject is pregnant and, in preferred embodiments, is carrying an HPA-1a positive fetus. In particular embodiments, the subject is HLA-DRB3*01:01 positive.

In embodiments of the invention, administration of the anti-HPA-1a monoclonal antibody achieves clearance of HPA-1a positive platelets in the subject within about 10 hours, or within about 5 hours, of administering the anti-HPA-1a monoclonal antibody to the subject. In certain embodiments, administration of the anti-HPA-1a monoclonal antibody achieves clearance of HPA-1a positive platelets in the subject within about 3 hours of administering the anti-HPA-1a monoclonal antibody to the subject.

In some embodiments, administration of the anti-HPA-1a monoclonal antibody prevents an alloimmune response to HPA-1a-positive platelets in the subject. In certain embodiments, administration of the anti-HPA-1a monoclonal antibody induces antibody-mediated immune suppression of an immune response to HPA-1a-positive platelets in the subject.

In some embodiments, half-life of the HPA-1a positive platelets is reduced by about 150-fold to about 250-fold in the subject relative to a subject who has not been administered the anti-HPA-1a monoclonal antibody.

In embodiments of the invention, the pharmaceutical composition further comprises succinate, arginine, polysorbate 80, and water for injection. In certain embodiments, the pharmaceutical composition has a pH of about 6.0-6.5.

Further aspects, features, and advantages of the present invention will be better appreciated upon a reading of the following detailed description of the invention and claims.

### BRIEF DESCRIPTION OF THE FIGURES

FIG. 1A-1C show the mean (±standard deviation) observed serum concentrations after a single SC dose of 0.21 mg of RLYB212 (n=6). For ease of interpretation, the datasets are plotted on linear-linear axes in FIG. 1A and FIG. 1B (on different time scales), as well as on log-linear axes in FIG. 1C.

FIG. 2A-2B show results of platelet saturation studies. FIG. 2A shows flow cytometric analysis of the binding of human IgG (left panel) or RLYB212 (right panel) to isolated human platelets from donors heterozygous for HPA-1. FIG. 2B shows flow cytometry results of two experiments in which HPA-1a/b platelets were incubated with the indicated antibody concentration in RLYB212, measured in mean fluorescence intensity (MFI).

### DETAILED DESCRIPTION OF THE INVENTION

The practice of the present invention will employ, unless otherwise indicated, conventional techniques of pharmaceutics, formulation science, immunology, hematology, cell biology, molecular biology, clinical pharmacology, and clinical practice, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Wherever embodiments are described with the language “comprising” or “having,” otherwise analogous embodiments described in terms of “consisting of” and/or “consisting essentially of” are included.

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10, from 1-8, from 3-9, and so forth. Likewise, a disclosed range is a disclosure of each individual value encompassed by the range. For example, a stated range of 5-10 is also a disclosure of 5, 6, 7, 8, 9, and 10. Where a numeric term is preceded by “about,” the term includes the stated number and values ±10% of the stated number.

The term “antibody” refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. The terms “antibody” or “immunoglobulin” are used interchangeably herein.

An antibody is typically composed of two identical pairs of polypeptide chains, each pair having one “heavy” chain and one “light” chain. Each chain is comprised of a variable region, which forms the antibody binding site, and a constant region, which may mediate the binding of the antibody to host tissues or factors. Immunoglobulin molecules can be divided into subclasses depending on the constant region of the heavy chain. The classes are immunoglobulin gamma (IgG), immunoglobulin mu (IgM), immunoglobulin delta (IgD), immunoglobulin epsilon (IgE), and immunoglobulin alpha (IgA). The heavy chain constant regions differ structurally and antigenically among the classes. IgG is the main type of antibody found in blood and extracellular fluid, and it plays a central role in the humoral immune response.

A “monoclonal antibody” (mAb) refers to a homogeneous antibody population that is involved in the highly specific recognition and binding of a single antigen binding site (epitope). The antigen-binding site can be defined using various terms and numbering schemes, including the following:
(i) Kabat. In the Kabat scheme, CDRs are based on sequence variability (Wu and Kabat 1970). Generally, the antigen-binding site has three CDRs in each variable region (e.g., HCDR1, HCDR2 and HCDR3 in the heavy chain variable region (VH) and LCDR1, LCDR2 and LCDR3 in the light chain variable region (VL));
(ii) Chothia. In the Chothia scheme, the term “hypervariable region” refers to the regions of an antibody variable domain that are hypervariable in structure as defined by Chothia and Lesk (Chothia and Lesk 1987). Generally, the antigen-binding site has three hypervariable regions in each VH (H1, H2, H3) and VL (L1, L2, L3). Numbering systems as well as annotation of CDRs and HVs have been revised by Abhinandan and Martin (Abhinandan and Martin 2008).
(iii) IMGT. The IMGT scheme is based on the comparison of variable domains from immunoglobulins and T-cell receptors. The International ImMunoGeneTics (IMGT) database provides a standardized numbering and definition of these regions. The correspondence between CDRs, HVs and IMGT delineations is described in Lefranc et al. (2003).
(iv) AbM. The AbM scheme is a compromise between the Kabat and Chothia numbering schemes and is described by Martin (2010).
(v) SDRU. The antigen-binding site can also be delineated based on “Specificity Determining Residue Usage” (SDRU) (Almagro 2004), where SDR refers to amino acid residues of an immunoglobulin that are directly involved in antigen contact.

Glycoprotein IIb/IIIa (GPIIb/IIIa), also known as integrin αIIbβ3, is a platelet membrane glycoprotein that binds fibrinogen and von Willebrand factor, and plays a role in platelet activation. HPA-1 is a polymorphism at position 33 on the mature β3 chain of GPIIb/IIIa. In particular, individuals who have a Leu at position 33 in one or more copies of ITGB3 (i.e., the gene that encodes integrin β3) or in any of their integrin β3 are “HPA-1a positive,” “positive for HPA-1a,” or “HPA-1a,” while individuals who do not have a Leu at position 33 (e.g., have a Pro at position 33) in all copies of ITGB3 or in all of their integrin β3 are “HPA-1a negative” or “negative for HPA-1a.”

Anti-HPA-1a antibodies for use in the invention are “specific for HPA-1a” or “specifically binds to HPA-1a,” which means that they do not display detectable binding to HPA-1b.

The term “pharmaceutical composition” refers to a preparation that is in such form as to permit the biological activity of the active ingredient to be effective and which contains no additional components that are unacceptably toxic to a subject to which the composition would be administered. Such composition can be sterile and can comprise a pharmaceutically acceptable carrier, such as physiological saline. Suitable pharmaceutical compositions can comprise one or more of a buffer (e.g., acetate, phosphate, or citrate buffer), a surfactant (e.g., polysorbate), a stabilizing agent (e.g., polyol or amino acid), a preservative (e.g., sodium benzoate), and/or other conventional solubilizing or dispersing agents.

A “subject” or “individual” or “patient” is any subject, particularly a mammalian subject, for whom diagnosis, prognosis, or therapy is desired. Mammalian subjects include humans, domestic animals, farm animals, sports animals, and laboratory animals including, e.g., humans, non-human primates, canines, felines, porcines, bovines, equines, rodents, including rats and mice, rabbits, etc.

The term “international unit” or “IU” is a unit of measurement of the amount of a substance as determined by its activity. The mass or volume that constitutes one international unit of a substance will vary based on the substance that is being measured. For anti-HPA-1a antibodies, the amount of anti-HPA1a antibodies in one IU is set to an international standard (Allen et al. 2005) adopted by the World Health Organization (see WHO International Standard: Anti-HPA-1a Standard (100 IU)).

As used herein, “WHO Standard” refers to the WHO International Standard of anti-HPA-1a antibodies, prepared by pooling human plasma collected from six donors immunized against HPA-1a (see WHO International Standard: Anti-HPA-1a Standard (100 IU)).

An “effective amount” of a therapy is an amount sufficient to carry out a specifically stated purpose, such as to elicit a desired biological or medicinal response in a subject.

“Pharmacokinetics” or “PK” refers to the study of how an administered agent is processed by the body of a subject. PK determinations include how the agent enters the blood circulation (absorption), is dispersed or disseminated throughout the fluids and tissues of the body (distribution), is recognized and transformed by the body (metabolism), and is removed from the body (excretion). The agent can be an active agent, e.g., a therapeutic antibody. Pharmacokinetics can be evaluated using various metrics, many of which are calculated based on the quantity of the agent in the body (e.g., in the plasma) at various time points following the administration of the agent.

“AUC” or “area-under-the-curve” is a pharmacokinetics metric that describes the variation of the concentration of an agent in blood plasma as a function of time. AUC may be calculated for different periods of time, for example, from time zero to specified time t (AUCₜ or AUC₀₋ₜ), from time zero to infinity (AUC_{∞} or AUC_{0-∞}), etc. “Cₘₐₓ” is the peak plasma concentration of an agent after administration. Time after administration is measured from T₀, which is the time that administration of a single dose of the agent is administered. “Tₘₐₓ” refers to the time of after administration of the agent (To) to reach maximum plasma concentration (Cₘₐₓ) of the agent. “T_{1/2}” refers to the half-life of the agent, i.e., the time required for the concentration of the agent to reach half of its original value. “CL/F” is the apparent clearance of the agent from plasma.

The terms “clear” or “clearance” or “eliminate” or “elimination” are used interchangeably and refer to achieving an undetectable level of a cell type, for example, HPA-1a positive platelets. Detection of the cell type can be carried out by known methods, including, for example, immunohistochemistry or flow cytometry, such as fluorescence-activated cell sorting (FACS).

“Binding” generally refers to the non-covalent interaction between a single binding site of a molecule and its binding partner (e.g., a receptor and its ligand, an antibody and its antigen, two monomers that form a dimer, etc.). In the case of binding between an antibody and its antigen, the interaction can, for example, prevent other molecules from binding to or recognizing the antigen, can initiate the destruction of the antigen, or can alter the structure or functionality of the antigen.

The terms “alloimmune response” or “alloimmunization” is an immune response to non-self antigens that are from the same species. As a result, the body produces antibodies against the non-self antigens.

### Monoclonal Anti-HPA-1a Antibodies

The invention relates to the use of a composition comprising a monoclonal antibody that specifically binds to anti-HPA-1a and comprises complementarity determining regions (CDRs) designated by the IMGT system as follows:
(a) a variable heavy (VH) CDR1 having the amino acid sequence of SEQ ID NO: 3,
(b) a VH CDR2 having the amino acid sequence of SEQ ID NO: 4,
(c) a VH CDR3 having the amino acid sequence of SEQ ID NO: 5,
(d) a variable light (VL) CDR1 having the amino acid sequence of SEQ ID NO: 6,
(e) a VL CDR2 having the amino acid sequence of SEQ ID NO: 7, and
(f) a VL CDR3 having the amino acid sequence of SEQ ID NO: 8.

In some embodiments, the anti-HPA-1a monoclonal antibody comprises a heavy chain variable region having the amino acid sequence of SEQ ID NO: 1, and a light chain variable region having the amino acid sequence of SEQ ID NO: 2.

In a particular embodiment, the anti-HPA-1a monoclonal antibody is a recombinant human immunoglobulin G1 monoclonal antibody that comprises a heavy chain having the amino acid sequence of SEQ ID NO: 8, and a light chain having the amino acid sequence of SEQ ID NO: 9, referred to herein as “RLYB212.”

In some embodiments, the anti-HPA-1a monoclonal antibody can be formulated in a pharmaceutical composition. The pharmaceutical composition may comprise one or more carriers, diluents, excipients, or other additives. For example, the composition can comprise one or more stabilizing agents (e.g., dextran 40, glycine, lactose, mannitol, trehalose, maltose, arginine), one or more buffers (e.g., acetate, citrate, histidine, lactate, phosphate, Tris), one or more pH adjusting agents (e.g., hydrochloric acid, nitric acid, potassium hydroxide, sodium hydroxide), one or more surfactants (polysorbate, sodium lauryl sulfate, polyethylene glycol-fatty acid esters, lecithins), and/or one or more diluents (e.g., water, physiological saline). The pH of the composition is preferably between about 4.0 and 8.0. In certain embodiments, the pH is between about 5.0 and 7.0, or between about 6.0 and 6.5, or about 6.3. In certain embodiments, the composition does not comprise any preservatives. In certain embodiments, the composition does not comprise mercury.

In a particular embodiment, the pharmaceutical composition comprises RLYB212, succinate, arginine, polysorbate 80, and water for injection.

The anti-HPA-1a monoclonal antibody can be prepared by methods known in the art. For example, the anti-HPA-1a monoclonal antibody can be prepared from memory B cells isolated from an HPA-1a alloimmunized subject according to the method described by Eksteen et al. (2015). Alternatively, a recombinant anti-HPA-1a monoclonal antibody, such as RLYB212, can be expressed in host cells. In another embodiment, the anti-HPA-1a monoclonal antibody can be raised in mice or other mammals immunized with human HPA-1a, produced using hybridomas, and humanized.

### Methods of Administering Anti-HPA-1a Monoclonal Antibodies

Aspects of the invention relate to methods of parenterally administering a monoclonal antibody that specifically binds HPA-1a, such as RLYB212, to a subject that is HPA-1a negative.

In some embodiments, the anti-HPA-1a monoclonal antibody is administered to the subject in an amount of about 0.03 mg to about 0.6 mg, or about 0.04 mg to about 0.4 mg, or about 0.05 mg to about 0.3 mg, or about 0.05 mg to about 0.1 mg, or about 0.1 mg to about 0.29 mg, or about 0.15 mg to about 0.25 mg. For example, the monoclonal antibody is administered in an amount of about 0.03 mg, or about 0.035 mg, or about 0.04 mg, or about 0.045 mg, or about 0.05 mg, or about 0.055 mg, or about 0.06 mg, or about 0.065 mg, or about 0.07 mg, or about 0.075 mg, or about 0.08 mg, or about 0.085 mg, or about 0.09 mg, or about 0.095 mg, or about 0.1 mg, or about 0.11 mg, or about 0.12 mg, or about 0.13 mg, or about 0.14 mg, or about 0.15 mg, or about 0.16 mg, or about 0.17 mg, or about 0.18 mg, or about 0.19 mg, or about 0.2 mg, or about 0.21 mg, or about 0.22 mg, or about 0.23 mg, or about 0.24 mg, or about 0.25 mg or about 0.29 mg, or any value in between. These amounts can also serve as endpoints for a range of amounts of monoclonal antibody to be administered, for example, about 0.06 mg to about 0.21 mg, or about 0.11 mg to about 0.17 mg, etc.

In some embodiments, the dose of the anti-HPA-1a monoclonal antibody is about 5 μg to about 400 μg or about 10 μg to about 300 μg, or about 15 μg to about 270 μg, or about 20 μg to about 250 μg, or about 25 μg to about 180 μg, or about 30, 35, 40, 45, 50, 55, 60, 65, 70, 75, 80, 85, 90, 100, 110, 120, 130, 140, 150, 160, 170, 180, 190, 200, 210, 220, 230, 240, 250, 260, 270, 280, 290, or 300 μg. In particular embodiments, the dose of the anti-HPA-1a monoclonal antibody is about 20 μg, about 40 μg, about 180 μg, or about 270 μg.

In some embodiments, the anti-HPA-1a monoclonal antibody is administered to the subject in an amount of about 1,500 IU to about 24,000 IU, or about 1,700 IU to about 18,000 IU, or about 30,000 IU to about 15,000 IU, or about 1,500 IU to about 6,000 IU, or about 6,000 IU to about 15,000 IU, or about 9,000 IU to about 15,000 IU. For example, the monoclonal antibody is administered in an amount of about 1,800 IU, or about 2,400 IU, or about 3,000 IU, or about 3,600 IU, or about 4,200 IU, or about 4,800 IU, or about 5,600 IU, or about 6,000 IU, or about 6,600 IU, or about 7,200 IU, or about 7,800 IU, or about 8,400 IU, or about 9,000 IU, or about 9,600 IU, or about 10,200 IU, or about 10,800 IU, or about 11,400 IU, or about 12,000 IU, or about 12,600 IU, or about 13,200 IU, or about 13,800 IU, or about 14,400 IU, or about 15,000 IU, or any value in between, including 12,390 IU. These amounts can also serve as endpoints for a range of amounts of monoclonal antibody to be administered, for example, about 1,800 IU to about 12,600 IU, or about 6,600 IU to about 10,800 IU, etc.

In certain embodiments, the dose of the anti-HPA-1a monoclonal antibody is about 300-24,000 IU or about 1,500-14,000 IU or about 1,800-11,000 IU or about 3,000-4,000 IU or about 3,500 IU or about 300-2,400 IU or about 350-2,100 IU or about 600-900 IU or about 700 IU.

In some embodiments, the anti-HPA-1a monoclonal antibody is administered in an amount effective to achieve a maximum plasma concentration of monoclonal antibody of about 3 ng/mL to about 40 ng/mL, or about 5 ng/mL to about 30 ng/mL, or about 5 ng/mL to about 25 ng/mL, or about 6 ng/mL to about 10 ng/mL, or about 3 ng/mL to about 10 ng/mL, or about 7 ng/mL to about 9 ng/mL, or about 20 ng/mL to about 30 ng/mL, in the subject. For example, the monoclonal antibody is administered in an amount effective to achieve a maximum plasma concentration of about 3 ng/mL, or about 5 ng/mL, or about 6 ng/mL, or about 7 ng/mL, or about 8 ng/mL, or about 9 ng/mL, or about 10 ng/mL, or about 11 ng/mL, or about 12 ng/mL, or about 13 ng/mL, or about 14 ng/mL, or about 15 ng/mL, or about 16 ng/mL, or about 17 ng/mL, or about 18 ng/mL, or about 19 ng/mL, or about 20 ng/mL, or about 21 ng/mL, or about 22 ng/mL, or about 23 ng/mL, or about 24 ng/mL, or about 25 ng/mL, or about 26 ng/mL, or about 27 ng/mL, or about 28 ng/mL, or about 29 ng/mL, or about 30 ng/mL, or any value therebetween, including 7.5 ng/mL or 8.5 ng/mL, in the subject. These amounts can also serve as endpoints for a range of maximum plasma concentrations of monoclonal antibody to be achieved in the subject, for example, about 7 ng/mL to about 26 ng/mL, or about 10 ng/mL to about 20 ng/mL, etc. In a preferred embodiment, the anti-HPA-1a monoclonal antibody is administered in an amount effective to achieve a maximum plasma concentration of monoclonal antibody of about 8 ng/mL or about 8.5 ng/mL or about 25 ng/mL.

In some embodiments, the anti-HPA-1a monoclonal antibody is administered in an amount effective to achieve a maximum plasma concentration of about 0.2 IU/mL to about 2.5 IU/mL, or about 0.2 IU/mL to about 2 IU/mL, or about 0.3 IU/mL to about 1.5 IU/mL, or about 0.3 IU/mL to about 0.7 IU/mL, or about 0.4 IU/mL to about 0.5 IU/mL, or about 1.2 IU/mL to about 1.8 IU/mL, in the subject. For example, the monoclonal antibody is administered in an amount effective to achieve a maximum plasma concentration of about 0.2 IU/mL, or about 0.3 IU/mL, or about 0.4 IU/mL, 0.5 IU/mL, or about 0.6 IU/mL, or about 0.7 IU/mL, or about 0.8 IU/mL, or about 0.9 IU/mL, or about 1 IU/mL, or about 1.1 IU/mL, or about 1.2 IU/mL, or about 1.3 IU/mL, or about 1.4 IU/mL, or about 1.5 IU/mL, or about 1.6 IU/mL, or about 1.7 IU/mL, or about 1.8 IU/mL, or about 1.9 IU/mL, or about 2 IU/mL, or any value therebetween, such as 0.45 IU/mL or 1.48 IU/mL, in the subject. These amounts can also serve as endpoints for a range of maximum plasma concentrations of monoclonal antibody to be achieved in the subject by administering RLYB212, for example, about 0.4 IU/mL to about 1.5 IU/mL, or about 0.6 IU/mL to about 1.2 IU/mL, etc.

In some embodiments, the anti-HPA-1a monoclonal antibody is administered as more than one dose. For example, the monoclonal antibody can be administered weekly. Weekly administration can be repeated, for example, for a total of 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks. In some embodiments, repeated doses of the anti-HPA-1a monoclonal antibody are administered biweekly (Q2W), for example, for a total of 2, 3, 4, 5, 6, 7, 8, 9, 10, 11, 12, 13, 14, 15, or 16 doses. In certain embodiments, the initial dose of the anti-HPA-1a monoclonal antibody is higher than subsequent doses.

In embodiments of the invention, administration of the anti-HPA-1a monoclonal antibody achieves clearance of HPA-1a positive platelets in the subject. HPA-1a positive platelets may be present in the subject for reasons that include, but are not limited to, the subject received a transfusion of HPA-1a positive platelets, and HPA-1a positive platelets were introduced to the subject during pregnancy (e.g., fetal HPA-1a positive platelets entered the subject's circulation).

In some embodiments, administration of the anti-HPA-1a monoclonal antibody achieves accelerated clearance of HPA-1a positive platelets in the subject or in a population of subjects. For example, clearance can be achieved in a subject or mean clearance can be achieved in a population of subjects within about 10 hours of administering the anti-HPA-1a monoclonal antibody, such as within about 10 hours, or about 9 hours, or about 8 hours, or about 7 hours, or about 6 hours, or about 5 hours, or about 4 hours, or about 3 hours, or about 2 hours, or about 1 hour, of administering the anti-HPA-1a monoclonal antibody to the subject(s). In some embodiments, clearance can be achieved in a subject or mean clearance can be achieved in a population of subjects within about 1 hour to about 10 hours, or about 1 hour to about 5 hours, or about 2 hours to about 4 hours, or about 2 hours to about 3 hours, of administering the anti-HPA-1a monoclonal antibody to the subject(s). In a preferred embodiment, the clearance or mean clearance is within about 2 hours of administering the anti-HPA-1a monoclonal antibody to the subject(s).

In some embodiments, administration of the anti-HPA-1a monoclonal antibody according to the present invention prevents an alloimmune response to HPA-1a-positive platelets in the subject. In some embodiments, administration of the anti-HPA-1a monoclonal antibody according to the present invention induces antibody-mediated immune suppression of an immune response to HPA-1a-positive platelets in the subject. In some embodiments, half-life of the HPA-1a positive platelets is reduced by about 150-250 fold in the subject, relative to a subject who has not been administered the anti-HPA-1a monoclonal antibody. In certain embodiments, mean half-life of the HPA-1a positive platelets is about 15, 20, 25, 30, 35, 40, 45, 50, or 55 minutes; or about 0.2, 0.3, 0.4, 0.5, 0.6, 0.7, 0.8, 0.9, or 1 hour, or about 2, 3, 4, 5, 6, 7, 8, 9, 10, 11, 12, 13, 14, 15, 16, 17, 18, 19, 20, 21, 22, 23, or 24 hours.

The pharmaceutical composition comprising the monoclonal anti-HPA-1a antibody is administered parenterally. Parenteral routes of administration include intravenous, intramuscular, intraperitoneal, intrathecal, and subcutaneous. In a preferred embodiment, the pharmaceutical composition is administered subcutaneously.

In embodiments of the invention, the subject is a woman who is HPA-1a negative and is pregnant. In some embodiments, the subject is a woman who is HPA-1a negative and had a previous pregnancy in which the fetus was HPA-1a positive. In other embodiments, the subject is a pregnant woman who is HPA-1a negative and is carrying an HPA-1a positive fetus.

In some embodiments, the subject may carry the HLA-DRB3*01:01 allele, i.e., is HLA-DRB3*01:01 positive. In other embodiments, the subject lacks the HLA-DRB3*01:01 allele.

As provided herein, the pharmaceutical composition may be administered without inducing a severe adverse event in the subject. As used herein, a severe adverse event encompasses any event that is fatal or immediately life-threatening; that requires inpatient hospitalization or prolongation of existing hospitalization; that results in persistent disability/incapacity; or that is a congenital anomaly/birth defect.

### EXAMPLES

Embodiments of the present disclosure can be further defined by reference to the following non-limiting examples. It will be apparent to those skilled in the art that many modifications, both to materials and methods, can be practiced without departing from the scope of the present disclosure.

### Example 1: RLYB212 Pharmacokinetics

A randomized, multi-center, placebo-controlled, single-blind study is conducted to evaluate the safety, immunogenicity, and pharmacokinetics of RLYB212 in healthy HPA-1a negative (HPA-1b/b) subjects.

### Objectives and Endpoints

The objectives and endpoints of the study are presented in Table 1.

TABLE 1
Objectives and endpoints of the study.

| Objectives | Endpoints |
|---|---|
| Primary objective: to evaluate the safety | type, seriousness, and incidence of adverse of |
| of single and multiple doses of RLYB212 | events |
| | vital signs |
| | clinical laboratory values |
| | electrocardiogram (ECG) |
| Secondary objective: to evaluate the | anti-drug antibodies (ADA) |
| immunogenicity of single and multiple | |
| doses of RLYB212 | |
| Secondary objective: to establish the | PK parameters of RLYB212 |
| pharmacokinetic profile of RLYB212 | half-life (t_{1/2}) of RLYB212 |
| following subcutaneous administration | maximum concentration (Cₘₐₓ) of |
| | RLYB212 |
| | time to maximum concentration (Tₘₐₓ) |
| | of RLYB212 |
| | apparent clearance (CL/F) of RLYB212 |
| | area under the concentration (AUC) of |
| | RLYB212 versus time curve |

### Overall Design

The study consists of two cohorts (1 and 2), with participants in each cohort randomized 3:1 to receive RLYB212 or a placebo, in a blinded manner. Volunteers initially entered a screening period (up to 42 days) to identify eligible participants. The participants then entered the study treatment period, in which the participants in both cohorts receive the study drug. Participants in Cohort 1 received a dose of the study drug on Day 1 only. Participants in Cohort 2 receive a dose of the study drug on Day 1 followed by 5 biweekly doses following the Day-1 dose. The post-treatment follow-up period continues for up to 12 weeks after the last dose is administered.

Data from both cohorts are used to establish RLYB212 safety, immunogenicity, and PK. For the first four participants in each cohort, the randomization is arranged to include one placebo participant as the first or second participant and one placebo participant as the third or fourth participant, while the remaining four participants receive RLYB212.

### Study Duration

The study duration is approximately 18 weeks for an individual participant in Cohort 1, and approximately 30 weeks for an individual participant in Cohort 2. These durations include a screening period of up to 6 weeks.

### Study Drug and Administration

Administration is as follows:
Cohort 1: 0.21 mg of RLYB212 or placebo on Day 1
Cohort 2: 0.29 mg of RLYB212 or placebo on Day 1, followed by 5 maintenance doses of 0.1 mg RLYB212 every two weeks (Q2W).

RLYB212 and placebo are administered subcutaneously. The placebo is a sodium chloride injection, 0.9% (saline).

### Analyses

Safety is analyzed through summaries of adverse events and severe adverse events, clinical laboratory evaluations (hematology, serum chemistry, coagulation, and urinalysis), ECGs, vital signs, and endogenous anti-HPA-1a antibodies.

Study drug exposure is analyzed through calculations of serum PK parameters, including AUC (extrapolated to infinity and/or over the dosing interval), Cₘₐₓ, Tₘₐₓ, CL/F, and t_{1/2}. Serum samples are collected for measurement of study drug concentrations on Days 1, 4, 8, 15, 29, 57, and 78 for Cohort 1 participants; and on Days 1, 4, 8, 15, 22, 29, 36, 43, 50, 57, 64, 71, 78, and 81 for Cohort 2 participants.

The immunogenicity analysis involves evaluating serum samples collected from the participants and screening the samples for antibodies binding to RLYB212. The titer of confirmed positive samples is also assessed. The analysis may further involve calculating percentage of participants with positive anti-drug antibodies (ADA) and ADA titers as applicable.

### Results

Cohort 1 consisted of 6 healthy, HPA-1a-negative, women and men. Following subcutaneous injection of 0.21 mg RLYB212 or placebo on Day 1, subjects submitted to serial blood sampling for up to 85 days. RLYB212 serum concentrations were determined using a validated enzyme-linked immunosorbent assay (ELISA) assay (lower limit of quantitation [LLOQ]: 1 ng/mL). Noncompartmental PK analysis (NCA), estimating exposure metrics (cₘₐₓ [the highest serum concentration] and tₘₐₓ, [time of peak serum concentration] AUC_{∞} [total area under the curve from time zero to infinity] and t_{1/2}^{term} [terminal half-life]). PK analysis was performed using actual sampling times for each of the 6 subjects.

Mean, standard deviation (SD), coefficient of variation (COV) as well as minimum and maximum value were calculated to characterize the cohort (n=6). Coefficient of determination (r²) was used as goodness-of-fit criteria for both the loglinear regression of the terminal portion as part of NCA as well as any of the compartmental PK model fits.

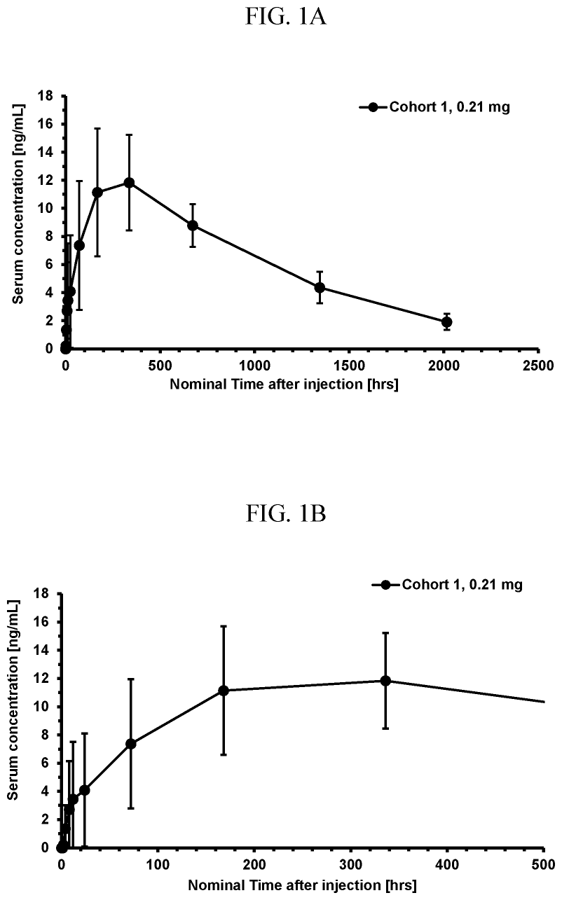

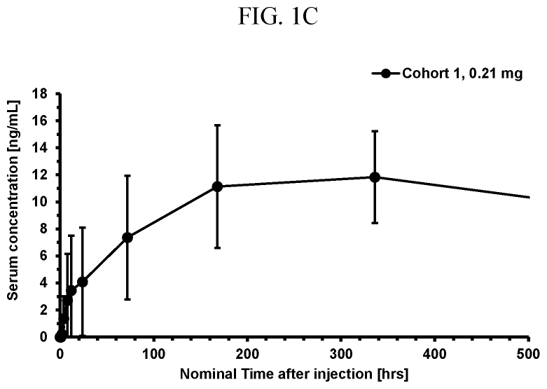

Results are shown in FIG. 1A-1C. Individual values for c max (mean: 12.3±3.9 ng/mL) and tₘₐₓ (mean: 10.9±3.6 days) following SC administration of 0.21 mg showed moderate inter-subject variability of about 30%. None of the cₘₐₓ values exceeded the pre-specified safety threshold of 25 ng/mL. AUC_{∞} (mean: 14,962 ng/mL*hrs) showed slightly less population variability of 26%, while terminal half-life estimates (mean: 25.4 days) were very similar across patients with a COV of only 8%. These findings suggest that SC absorption is a source of observed PK variability, while systemic disposition is quite consistent among patients.

### Example 2: RLYB212 Platelet Elimination

RLYB212 is analyzed for its capacity to eliminate HPA-1a positive platelets transfused to HPA-1a negative (HPA-1b/b) healthy male volunteers.

### Objectives and Endpoints

The objectives and endpoints of the study are presented in Table 2.

TABLE 2
Objectives and endpoints of the study.

| Objectives | Endpoints |
|---|---|
| Primary objective: to establish the ability | half-life (t_{1/2}) of transfused platelets |
| of RLYB212 to markedly (10-fold or | |
| greater) accelerate the elimination of HPA- | |
| 1a positive platelets transfused to HPA- | |
| 1b/b healthy volunteers | |
| Secondary objective: to evaluate the | type, seriousness, and incidence of adverse |
| safety of a single dose of RLYB212 | events |
| | vital signs |
| | clinical laboratory values |
| | ECG |
| Secondary objective: to monitor | development of anti-HPA-1a alloantibodies |
| alloimmune response to HPA-1a positive | |
| platelets | |
| Exploratory objective: to evaluate the | ADAs |
| immunogenicity of a single dose of | |
| RLYB212 | |

### Overall Design

The study consists of one cohort of eight participants that are randomized 3:1 to receive RLYB212 or placebo in a single-blinded manner. Volunteers initially enter a screening period (up to 42 days) to identify eligible participants. The participants then enter the study treatment period, in which the participants receive the study drug (RLYB212 or placebo). For the first four participants, the randomization is arranged to include one placebo participant as the first or second participant, and one placebo participant as the third or fourth participant, while the remaining four participants receive RLYB212.

### Study Duration

The study duration is approximately 18 weeks, ending 77 days after the transfusion of the platelets. This duration includes a screening period of up to 6 weeks.

### Study Drug and Platelet Administration

Participants are administered 0.09 mg RLYB212. The dose of RLYB212 is chosen to achieve a target peak plasma concentration of <0.45 IU/mL or 8 ng/mL, and is based in part on PK/PD modeling that incorporates the PK data obtained in Example 1. RLYB212 and placebo are administered subcutaneously. The placebo is a sodium chloride injection, 0.9% (saline).

Participants receive study drug on Day 1, and receive a transfusion of HPA-1a positive platelets on Day 8. HPA-1a platelet elimination is assessed through Day 15. The post-treatment follow-up period continues for up to 12 weeks after the last dose is administered. The platelet transfusion on Day 8 delivers 10×10⁹ HPA-1a positive platelets to the participants. The platelets are obtained from a donor who is HPA-1a/b positive.

### Platelet and Plasma Preparation

Platelets to be transfused are obtained by plateletpheresis from existing ABO-compatible platelet donors. Platelets are obtained 20 to 24 hours before transfusion. All platelet donors are HPA-1a/b heterozygous and HLA-A2 homozygous. None of the platelet donors have HLA antibodies.

Platelet-rich plasma prepared from whole blood collected from the recipients is used to determine the proportion of HLA-A2 positive platelets. Platelet-rich plasma is prepared from the anticoagulant citrate dextrose (ACD-A) plasma collected from study subjects, and platelets are immediately preserved with ThromboFix Platelet Stabilizer (Beckman Coulter) according to the manufacturer's instructions.

### Data Collection

The proportion of transfused HPA-1ab platelets in circulation after administration of RLYB212 or placebo is determined by flow cytometry using the HLA-A2 discrepancy between donor (HLA-A2 positive) and recipient (HLA-A2 negative). Proof of concept is prospectively defined as elimination of HPA-1a positive platelets by 10-fold or greater based on platelet half-life.

Study drug is administered 7 days before platelet transfusion. The elimination of transfused HPA-1a positive platelets is analyzed based on serum collected pre-transfusion and at 10, 20, 30, 40, and 50 minutes and 1, 2, 3, 4, and 24 hours after platelet transfusion; on day 3; and on day 7, if platelets are detected in a transfused individual on day 3.

### Flow Cytometry

A validated flow cytometry-based method is used to determine the frequency of HLA-A2 positive platelets in the recipient at specified time points after administration of RLYB212 (Vetlesen et al. 2012; Kjaer et al. 2018). Optimization and validation of this method are performed on mixtures of low frequencies of HLA-A2 positive platelets into HLA-A2 negative platelets, and expected frequencies are compared with observed frequencies. The lower limit of quantification (LLOQ) is 0.015%, and linearity is 0.97. Approximately 1.5×10⁶ platelets are double-stained with 6 fluorescein isothiocyanate (FITC)-conjugated HLA-A2 antibodies (3.6%; clone H0037; ONE Lambda Inc.) and 12 PCS-conjugated CD41 antibodies (0.7%; clone P2; Beckman Coulter). After 20 minutes of incubation in the dark at room temperature, 600 μL of fixation buffer (0.2% paraformaldehyde in phosphate-buffered saline) is added.

One million events are collected at the lowest collection rate using a Canto II flow cytometer (Becton Dickinson). FACSDiva software (BD Bioscience) is used to determine the proportion of transfused platelets. Platelets are identified on scatter plots showing the forward scatter properties versus PCS fluorescence, and the frequency of transfused platelets is assessed on scatter plots showing FITC fluorescence versus side scatter. Blood samples are analyzed consecutively. If transfused platelets cannot be detected in 2 consecutive samples, samples from subsequent time points are not obtained or examined.

### Statistical Analysis

Platelet kinetic analysis is performed on all subjects for whom sufficient data are available to derive at least one of the platelet kinetic endpoints. The safety analysis set includes all subjects who receive platelets and/or RLYB212 or placebo, and is used for reporting of safety, demographic characteristics, and exposure to treatment.

Individual plots of the primary endpoint are normalized to the baseline assessment, which is defined as the first post-infusion flow cytometry data point. Actual sampling time points relative to baseline are used for derivation of the noncompartmental analysis (NCA) and on the individual plots of platelet versus time. Flow cytometry-assessed platelet concentration values that are below the highest pre-baseline value are excluded from the NCA. Concentration values below the LLOQ and missing values are also excluded from the NCA. No formal analysis of “outliers” is performed.

For the primary endpoint, the terminal elimination half-life (t_{1/2}) rate is calculated by NCA. The elimination phase is determined by visual inspection of the individual concentration curves. At least 3 points above LLOQ belonging to the elimination phase are used for estimating the slope (λ_{z}) of the log concentration-versus-time curve.

### Secondary Analyses

Safety is analyzed through summaries of adverse events and severe adverse events, clinical laboratory evaluations (hematology, serum chemistry, coagulation, and urinalysis), ECGs, vital signs, and endogenous anti-HPA-1a antibodies.

The immunogenicity analysis involves evaluating serum samples collected from the participants and screening the samples for antibodies binding to RLYB212.

### Example 3: In Vitro Binding of RLYB212 to HPA-1a/b Platelets

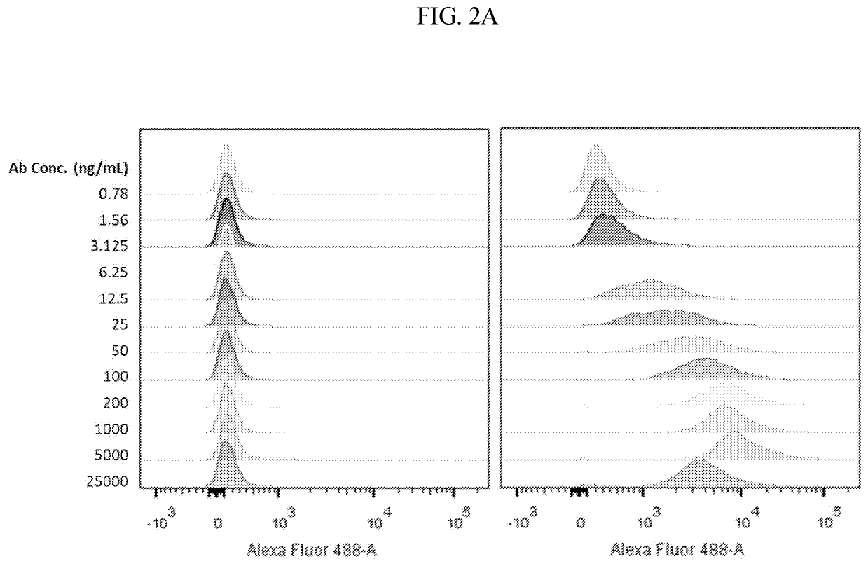

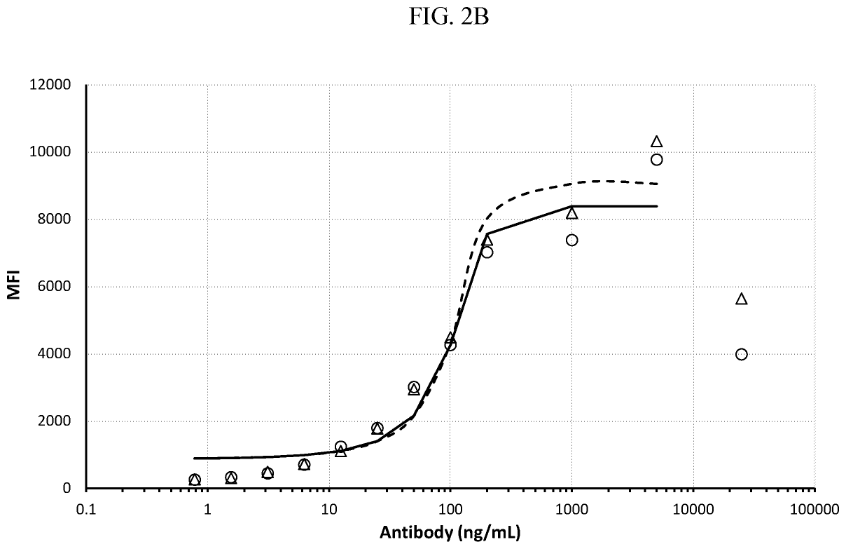

Human platelets heterozygous for HPA were incubated at a final concentration of 2×10⁶ cells/mL with human IgG1 (control) or RLYB212 at antibody concentrations of 0.78-25,000 ng/mL. Samples were washed in PBS buffer containing 1% bovine serum albumin. Washed platelets were incubated with fluorescein (FITC) AffiniPure F(ab′)₂ donkey anti-human IgG (Jackson ImmunoResearch Inc., West Grove, PA) and analyzed by flow cytometry. Data were analyzed using FlowJo software (Tree Star Inc., Ashland OR). Results are shown in FIG. 2A-2B. FIG. 2A represents histogram plots of Alexa Fluor signal at varying concentrations of human IgG1 control (left panel) or RLYB212 (right panel). FIG. 2B represents fitted sigmoidal curves of two replicates of the RLYB212 binding isotherm. Binding above the lower asymptote is detectable at approximately 5 ng/mL.

SEQUENCES

SEQ ID NO: 1 (RLYB212 Heavy Chain Variable Region)
Gln Val Gln Leu Gln Gln Ser Gly Pro Gly Leu Val Lys Pro Ser Gln Thr Leu Ser Leu Thr Cys
Ala Ile Ser Gly Asp Ser Val Ser Ser Asn Ser Ala Ala Trp Asn Trp Ile Arg Gln Ser Pro Ser Arg
Gly Leu Glu Trp Leu Gly Arg Thr Tyr Phe Arg Ser Asn Trp Tyr Asn Asp Tyr Ala Ala Ser Val
Lys Ser Arg Ile Thr Ile Asn Gln Asp Thr Ser Lys Asn Gln Leu Ser Leu Gln Leu Asn Ser Val Thr
Pro Glu Asp Thr Ala Val Tyr Tyr Cys Ala Arg Asp Gly Ala Trp Gly Gly Ser Ser Trp Trp Pro
Gly Leu Pro His His Tyr Tyr Ser Gly Met Asp Val Trp Gly Gln Gly Thr Thr Val Thr Val Ser Ser
SEQ ID NO: 2 (RLYB212 Light Chain Variable Region)
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg Leu
Leu Ile Tyr Asp Ala Ser Lys Arg Ala Thr Gly Ile Pro Ala Arg Phe Ser Gly Ser Gly Ser Gly Thr
Asp Phe Ser Leu Thr Ile Arg Ser Leu Glu Pro Glu Asp Phe Ala Val Tyr Tyr Cys Gln Gln Arg
Ser Asp Trp Gln Gly Leu Thr Phe Gly Gly Gly Thr Lys Val Glu Ile Lys
SEQ ID NO: 3 (RLYB212 Variable Heavy Complementarity Determining Region 1)
Gly Asp Ser Val Ser Ser Asn Ser Ala Ala
SEQ ID NO: 4 (RLYB212 Variable Heavy Complementarity Determining Region 2)
Thr Tyr Phe Arg Ser Asn Trp Tyr Asn
SEQ ID NO: 5 (RLYB212 Variable Heavy Complementarity Determining Region 3)
Ala Arg Asp Gly Ala Trp Gly Gly Ser Ser Trp Trp Pro Gly Leu Pro His His Tyr Tyr Ser Gly Met
Asp Val
SEQ ID NO: 6 (RLYB212 Variable Light Complementarity Determining Region 1)
Gln Ser Val Ser Ser Tyr
SEQ ID NO: 7 (RLYB212 Variable Light Complementarity Determining Region 2)
Asp Ala Ser
SEQ ID NO: 8 (RLYB212 Variable Light Complementarity Determining Region 3)
Gln Gln Arg Ser Asp Trp Gln Gly Leu Thr
SEQ ID NO: 9 (RLYB212 Heavy Chain)
Gln Val Gln Leu Gln Gln Ser Gly Pro Gly Leu Val Lys Pro Ser Gln Thr Leu Ser Leu Thr Cys
Ala Ile Ser Gly Asp Ser Val Ser Ser Asn Ser Ala Ala Trp Asn Trp Ile Arg Gln Ser Pro Ser Arg
Gly Leu Glu Trp Leu Gly Arg Thr Tyr Phe Arg Ser Asn Trp Tyr Asn Asp Tyr Ala Ala Ser Val
Lys Ser Arg Ile Thr Ile Asn Gln Asp Thr Ser Lys Asn Gln Leu Ser Leu Gln Leu Asn Ser Val Thr
Pro Glu Asp Thr Ala Val Tyr Tyr Cys Ala Arg Asp Gly Ala Trp Gly Gly Ser Ser Trp Trp Pro
Gly Leu Pro His His Tyr Tyr Ser Gly Met Asp Val Trp Gly Gln Gly Thr Thr Val Thr Val Ser Ser
Ala Ser Thr Lys Gly Pro Ser Val Phe Pro Leu Ala Pro Ser Ser Lys Ser Thr Ser Gly Gly Thr Ala
Ala Leu Gly Cys Leu Val Lys Asp Tyr Phe Pro Glu Pro Val Thr Val Ser Trp Asn Ser Gly Ala
Leu Thr Ser Gly Val His Thr Phe Pro Ala Val Leu Gln Ser Ser Gly Leu Tyr Ser Leu Ser Ser Val
Val Thr Val Pro Ser Ser Ser Leu Gly Thr Gln Thr Tyr Ile Cys Asn Val Asn His Lys Pro Ser Asn
Thr Lys Val Asp Lys Lys Val Glu Pro Lys Ser Cys Asp Lys Thr His Thr Cys Pro Pro Cys Pro
Ala Pro Glu Leu Leu Gly Gly Pro Ser Val Phe Leu Phe Pro Pro Lys Pro Lys Asp Thr Leu Met
Ile Ser Arg Thr Pro Glu Val Thr Cys Val Val Val Asp Val Ser His Glu Asp Pro Glu Val Lys Phe
Asn Trp Tyr Val Asp Gly Val Glu Val His Asn Ala Lys Thr Lys Pro Arg Glu Glu Gln Tyr Asn
Ser Thr Tyr Arg Val Val Ser Val Leu Thr Val Leu His Gln Asp Trp Leu Asn Gly Lys Glu Tyr
Lys Cys Lys Val Ser Asn Lys Ala Leu Pro Ala Pro Ile Glu Lys Thr Ile Ser Lys Ala Lys Gly Gln
Pro Arg Glu Pro Gln Val Tyr Thr Leu Pro Pro Ser Arg Asp Glu Leu Thr Lys Asn Gln Val Ser
Leu Thr Cys Leu Val Lys Gly Phe Tyr Pro Ser Asp Ile Ala Val Glu Trp Glu Ser Asn Gly Gln
Pro Glu Asn Asn Tyr Lys Thr Thr Pro Pro Val Leu Asp Ser Asp Gly Ser Phe Phe Leu Tyr Ser
Lys Leu Thr Val Asp Lys Ser Arg Trp Gln Gln Gly Asn Val Phe Ser Cys Ser Val Met His Glu
Ala Leu His Asn His Tyr Thr Gln Lys Ser Leu Ser Leu Ser Pro Gly Lys
SEQ ID NO: 10 (RLYB212 Light Chain)
Glu Ile Val Leu Thr Gln Ser Pro Ala Thr Leu Ser Leu Ser Pro Gly Glu Arg Ala Thr Leu Ser Cys
Arg Ala Ser Gln Ser Val Ser Ser Tyr Leu Ala Trp Tyr Gln Gln Lys Pro Gly Gln Ala Pro Arg Leu
Leu Ile Tyr Asp Ala Ser Lys Arg Ala Thr Gly Ile Pro Ala Arg Phe Ser Gly Ser Gly Ser Gly Thr
Asp Phe Ser Leu Thr Ile Arg Ser Leu Glu Pro Glu Asp Phe Ala Val Tyr Tyr Cys Gln Gln Arg
Ser Asp Trp Gln Gly Leu Thr Phe Gly Gly Gly Thr Lys Val Glu Ile Lys Arg Thr Val Ala Ala
Pro Ser Val Phe Ile Phe Pro Pro Ser Asp Glu Gln Leu Lys Ser Gly Thr Ala Ser Val Val Cys Leu
Leu Asn Asn Phe Tyr Pro Arg Glu Ala Lys Val Gln Trp Lys Val Asp Asn Ala Leu Gln Ser Gly
Asn Ser Gln Glu Ser Val Thr Glu Gln Asp Ser Lys Asp Ser Thr Tyr Ser Leu Ser Ser Thr Leu Thr
Leu Ser Lys Ala Asp Tyr Glu Lys His Lys Val Tyr Ala Cys Glu Val Thr His Gln Gly Leu Ser
Ser Pro Val Thr Lys Ser Phe Asn Arg Gly Glu Cys

### REFERENCES

Abhinandan K R and Martin A C R. Analysis and improvements to Kabat and structurally correct numbering of antibody variable domains. Mol. Immunol. 45: 3832-3839 (2008).
Allen D, et al. Collaborative study to establish the first international standard for quantitation of anti-HPA-1a. Vox Sang. 89: 100-104 (2005).
Almagro J C. Identification of differences in the specificity-determining residues of antibodies that recognize antigens of different size: implications for the rational design of antibody repertoires. Mol. Recognit. 17: 132-143 (2004).
Bussel J. Diagnosis and management of the fetus and neonate with alloimmune thrombocytopenia. J. Thromb. Haemost. 7: 253-257 (2009).
Chothia C and Lesk A M. Canonical structures for the hypervariable regions of immunoglobulins. J. Mol. Biol. 196: 901-917 (1987).
Commission E. Development of a prophylactic treatment for the prevention of fetal/neonatal alloimmune thrombocytopenia (FNAIT). PROFNAIT (2021).
Eksteen M, et al. Characterization of a human platelet antigen-1a-specific monoclonal antibody derived from a B cell from a woman alloimmunized in pregnancy. J. Immunol. 194: 5751-5760 (2015).
Kjaer M F E, et al. Establishment and validation of a laboratory assay for monitoring survival of transfused platelets: tracking HLA mismatch between donor and recipient. Vox Sanguinis 113(S1): 5-347 (2018).
Kjeldsen-Kragh J, et al. A screening and intervention program aimed to reduce mortality and serious morbidity associated with severe neonatal alloimmune thrombocytopenia. Blood 110: 833-9 (2007).
Kjeldesen-Kragh J and Skogen B. Mechanisms and prevention of alloimmunization in pregnancy. Obstet. Gynecol. Surv. 68: 526-532 (2013).
Kjeldsen-Kragh J and Olsen K J. Risk of HPA-1a-immunization in HPA-1a-negative women after giving birth to an HPA-1a-positive child. Transfusion 59: 1344-52 (2019).
Lefranc M-P, et al. IMGT unique numbering for immunoglobulin and T cell receptor variable domains and Ig superfamily V-like domains. Dev. Comp. Immunol. 27: 55-77 (2003).
Martin A C R. Antibody Engineering. Eds. Kontermann R, Dubel S (Springer-Verlag, Berlin). 2: 33-51 (2010).
Mortberg A, et al. Sensitive detection of platelet-specific antibodies with a modified MAIPA using biotinylated antibodies and streptavidin-coated beads. J Immunol Methods 434: 9-15 (2016).
Newman P J, et al. The human platelet alloantigens, Pl^{A1} and Pl^{A2}, are associated with a leucine³³/proline³³ amino acid polymorphism in membrane glycoprotein IIIa, and are distinguishable by DNA typing. J. Clin. Invest. 83: 1778-1781 (1989).
Radder C M, et al. Will it ever be possible to balance the risk of intracranial haemorrhage in fetal or neonatal alloimmune thrombocytopenia against the risk of treatment strategies to prevent it? Vox Sang. 84: 318-325 (2003).
Rossi K Q, et al. Effects of antepartum therapy for fetal alloimmune thrombocytopenia on maternal lifestyle. J. Matern. Fetal Neonatal Med. 29: 1783-1788 (2015).
Vitiello G, et al. Intravenous immunoglobulin therapy: a snapshot for the internist. Intern Emerg. Med. 14: 1041-1049 (2019).
WHO International Standard: Anti-HPA-1a Standard (100 IU). NIB SC code: 03/152. National Institute for Biological Standards and Control, Hertfordshire, United Kingdom.
Wu T T and Kabat E A, An analysis of the sequences of the variable regions of Bence Jones proteins and myeloma light chains and their implications for antibody complementarity. J. Exp. Med. 132: 211-250 (1970).
   The invention is further described by the following claims.

1. A method of administering an anti-human platelet antigen (HPA)-1a monoclonal antibody to a subject that is HPA-1a negative, the method comprising parenterally administering to the subject a pharmaceutical composition comprising the anti-HPA-1a monoclonal antibody;
wherein the anti-HPA-1a monoclonal antibody comprises (i) a variable heavy (VH) complementarity determining region (CDR) 1 having the amino acid sequence of SEQ ID NO: 3; (ii) a VH CDR2 having the amino acid sequence of SEQ ID NO: 4; (iii) a VH CDR3 having the amino acid sequence of SEQ ID NO: 5; (iv) a variable light (VL) CDR1 having the amino acid sequence of SEQ ID NO: 6; (v) a VL CDR2 having the amino acid sequence of SEQ ID NO: 7; and (vi) a VL CDR3 having the amino acid sequence of SEQ ID NO: 8; and
wherein the anti-HPA-1a monoclonal antibody is administered in an amount effective to achieve a maximum plasma concentration of the anti-HPA-1a monoclonal antibody of about 3 ng/mL to about 40 ng/mL in the subject.

2. The method of claim 1, wherein the anti-HPA-1a monoclonal antibody is administered in an amount of about 0.015 mg to about 0.6 mg.

3. The method of claim 1, wherein the anti-HPA-1a monoclonal antibody is administered in an amount of about 0.05 mg to about 0.35 mg.

4. The method of claim 1, wherein the maximum plasma concentration of the anti-HPA-1a monoclonal antibody is about 5 ng/mL to about 30 ng/mL.

5. The method of claim 1, wherein the maximum plasma concentration of the anti-HPA-1a monoclonal antibody is about 6 ng/mL to about 10 ng/mL.

6. The method of claim 1, wherein the maximum plasma concentration of the anti-HPA-1a monoclonal antibody is about 0.45 IU/mL.

7. The method of claim 1, wherein the pharmaceutical composition is administered subcutaneously.

8. The method of claim 1, wherein the subject is HPA-1a negative.

9. The method of claim 1, wherein the subject is pregnant.

10. The method of claim 1, wherein the subject is carrying an HPA-1a positive fetus.

11. The method of claim 1, wherein the subject is HLA-DRB3*01:01 positive.

12. The method of claim 1, wherein the anti-HPA-1a monoclonal antibody comprises a heavy chain variable region having the amino acid sequence of SEQ ID NO: 1, and a light chain variable region having the amino acid sequence of SEQ ID NO: 2.

13. The method of claim 1, wherein administration of the anti-HPA-1a monoclonal antibody achieves clearance of HPA-1a positive platelets in the subject within about 10 hours of administering the anti-HPA-1a monoclonal antibody to the subject.

14. The method of claim 1, wherein administration of the anti-HPA-1a monoclonal antibody achieves clearance of HPA-1a positive platelets in the subject within about 5 hours of administering the anti-HPA-1a monoclonal antibody to the subject.

15. The method of claim 1, wherein administration of the anti-HPA-1a monoclonal antibody achieves clearance of HPA-1a positive platelets in the subject within about 3 hours of administering the anti-HPA-1a monoclonal antibody to the subject.

16. The method of claim 1, wherein administration of the anti-HPA-1a monoclonal antibody prevents an alloimmune response to HPA-1a-positive platelets in the subject.

17. The method of claim 1, wherein administration of the anti-HPA-1a monoclonal antibody induces antibody-mediated immune suppression of an immune response to HPA-1a-positive platelets in the subject.

18. The method of claim 1, wherein half-life of the HPA-1a positive platelets is reduced by about 150-fold to about 250-fold in the subject relative to a subject who has not been administered the anti-HPA-1a monoclonal antibody.

19. The method of claim 1, wherein the pharmaceutical composition further comprises succinate, arginine, polysorbate 80, and water for injection.

20. The method of claim 1, wherein the pharmaceutical composition has a pH of about 6.0-6.5.
